(19) United States
(12) Patent Application Publication
(10) Pub. No.: US 2010/0221791 A1
(43) Pub. Date: Sep. 2, 2010

(54) Method for Incorporating Proteins Into Lentivirus Vectors

(76) Inventors: Diana Schenkwein, Kuopio (FI); Seppo Yla-Herttuala, Kuopio (FI)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/525,791

(22) PCT Filed: Feb. 11, 2008
(86) PCT No.: PCT/GB08/00465

§ 371 Date: May 10, 2010

(30) Foreign Application Priority Data
Feb. 12, 2007 (GB) 0702694.1

(51) Int. Cl. C12N 15/64

(52) U.S. Cl. 435/91.41

(57) ABSTRACT

The present invention is a method for incorporating an integrase-fusion protein into a third-generation lentivirus vector, comprising: (i) transfecting a vector packaging plasmid into a producer cell, wherein the vector packaging plasmid contains a lentivirus transfer construct and a gene encoding the integrase-fusion protein, said gene being fused to the pol-polyprotein gene; (ii) transcription and translation of the genes; and (iii) release of the integrase-fusion protein from the pol-polyprotein.

### FIELD OF THE INVENTION

This invention relates to a method for the incorporation of foreign proteins and integrase-fusion proteins into third generation HIV-1 based lentivirus vectors.

### BACKGROUND OF THE INVENTION

Integration of the viral cDNA into the cellular chromatin is a crucial step in the life cycle of both simple and complex retroviruses. This step makes these viruses attractive candidates as gene therapy vectors when long-term gene transfer is desired. Retroviral DNA integration is, however, a non-specific event that may cause insertional mutagenesis and lead to variations in cellular gene expression. Recently, leukemia cases have been linked to the use of retroviral vectors in gene therapy trials.

One approach to alleviate the problems arising from unspecific retroviral integration is to pursue targeted integration by integrase (IN) fusion proteins. The retroviral integrase, responsible for cDNA integration, can be fused to a sequence-specific DNA-binding protein, which is able to target a predefined chromosomal site (Bushman, 1994; Bushman, 1995; Goulaouic & Chow, 1996). It has been shown in in vitro studies, and in cell culture assays, that these IN-fusion proteins are capable of directing IN-mediated integration into, or close to, the predetermined sites recognized by the DNA binding proteins. However, efficient incorporation of IN-fusion proteins into gene therapy grade vectors has not been previously demonstrated.

Previously, IN-fusion proteins have been incorporated into lentivirus virions using a “trans-packaging” strategy (Wu et al., 1995; Fletcher et al., 1997). This method involves the HIV-1 accessory protein, Vpr, which is packaged into viruses by interacting with the p6 protein of Gag (Kondo et al, 1995). Co-expression of the Vpr fusion proteins and a HIV-1 molecular clone, by a producer cell-line, results in incorporation of the fusion protein into the newly forming virus (see FIG. 2). After packaging, Vpr is removed from the IN-fusion protein by HIV-1 protease (PR) (Fletcher et al., 1997).

The Vpr-mediated trans-packaging method has been used to package IN-fusion proteins into infectious HIV-1 virions, and also to target therapeutic proteins into nucleic acid, free protein-transducing nanoparticles (PTNs), based on lentiviral vectors (Tan et al., 2006; Holmes-Son & Chow., 2002). The trans-packaging method, however, has a number of disadvantages, such as the incomplete release of trans-packaged proteins from Vpr and unspecific cleavage of the incorporated fusion proteins. Further, incorporation of a proapoptotic protein into PTN particles failed, using the trans-packaging method (Link et al., 2006).

An alternative to the Vpr-dependent trans-packaging method is to incorporate IN-fusion proteins into virions by providing the fusion genes directly from the viral genome. Previous attempts to incorporate HIV-1 IN or avian sarcoma virus (ASV) IN fusion proteins into virus genomes resulted in loss of viral infectivity (Bushman & Miller, 1997) or loss of fusion protein expression during viral replication (Katz et al., 1996).

Third generation HIV-1 derived vectors are promising tools for gene therapy as they are able to infect both dividing and non-dividing cells, confer long-term gene transfer, can carry a reasonably large transgene cargo and are relatively easy to produce in high titers. Third generation HIV-1 derived vectors resemble their wild-type counterparts only in the early steps of the virus life cycle, ending in the integration of a transgene construct into the target cells chromatin.

Third generation HIV-1 based lentivirus (LV) vectors are produced by the co-transfection of four different plasmids encoding the structural and regulatory products needed to produce replication incompetent vector particles. The HIV-1 accessory genes, vif, vpr, vpu, and nef, have proven to be dispensable for vector production and have been deleted from packaging constructs. These genes are critical for the virus replication and pathogenesis in vivo but not crucial for viral growth or transduction in vitro. Vectors are not designed to replicate in target cells, but to only carry out the steps up to, and including, transgene integration.

### SUMMARY OF THE INVENTION

The present invention is a new method for incorporating IN-fusion proteins into self-inactivating third generation HIV-1 based lentivirus vectors. This is achieved by the expression of the fusion genes from the virus packaging plasmids as fusions to the pol gene. It is therefore independent of PR-mediated release of functional IN fusion proteins from Vpr, unlike the traditional trans-packaging method.

According to the present invention, a method for incorporating an integrase-fusion protein into a third-generation lentivirus vector, comprises:

(i) transfecting a vector packaging plasmid into a lentivirus producer cell, a gene encoding the integrase-fusion protein, said gene being fused to the pol-polyprotein gene;

(ii) transcription and translation of the genes by the lentivirus producer cell to produce a lentiviral vector particle; and

(iii) inside the vector particle, release of the integrase-fusion protein from the pot-polyprotein.

### DESCRIPTION OF THE DRAWINGS

The following drawings illustrate embodiments of the present invention.

FIG. 1 shows the packaging plasmid pMDLg/pRRE (left) and the IN-fusion gene containing packaging plasmid pMDLg/pRRE (right). The packaging plasmid can contain any IN-fusion protein cDNA. The enzymes sites used for cloning of the IN-fusion containing packaging plasmids are indicated. IN: integrase; PC: protease cleavage site; POL: HIV-1 polyprotein.

FIG. 2 shows a schematic presentation of vector production using the Vpr-mediated trans-packaging (A) and the modified 3^{rd} generation LVV production system (B) to package IN-fusion proteins into virions. The reference numbers represent the following stages of virus production:

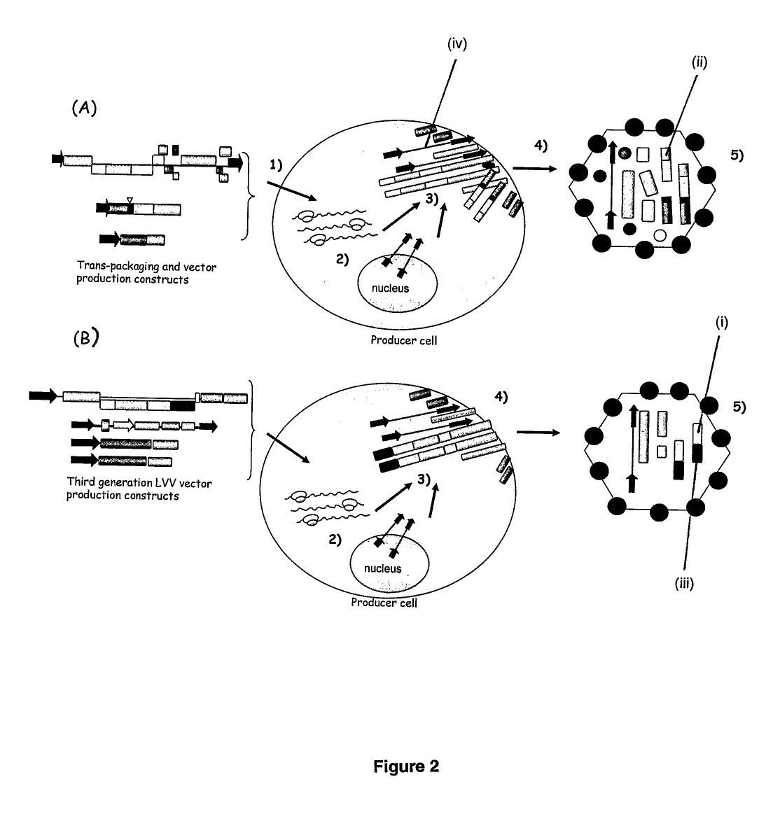

1) Vector producing plasmids are transfected into the producer cells;

2) Transcription and translation of the structural genes occurs in the producer cells. The LTR-flanked vector RNA (transfer construct) is also transcribed;

3) Structural proteins and the vector RNA assemble on the plasma membrane. The Vpr-IN-fusion proteins attach to the p6 region of the Gag protein (A);

4) Virions release by budding and PR mediated proteolytic maturation takes place. During maturation, polyproteins are cleaved to their individual fragments to release functional proteins;

5) A schematic presentation of the composition of the virions after partial maturation. A part of the Vpr-IN-fusion proteins remains unprocessed and a part of the functional IN-fusion proteins become released (A). In (B) IN-fusion proteins are effectively released from the pol-polyprotein:

(a) The packaging construct (b) Vpr-PC-IN-fusion protein construct (c) The Env-construct (pseudotyping) (d) Third generation LVV transfer construct (e) RRE-expression construct.

In the drawing, the reference numerals represent the following constructs: IN-proteins: (i); Vpr-PC-IN-fusion proteins: (ii); protein fused to IN: (iii); viral RNA genome (transfer construct): (iv).

### DESCRIPTION OF PREFERRED EMBODIMENTS

As used herein, the term integrase-fusion protein means a protein, which is fused to a viral integrase. The protein may be, for example, an endonuclease such as I-Ppol.

The term “lentivirus producer cell” will be known to those skilled in the art. A lentivirus producer cell is a producer cell capable of producing lentiviruses. In the method of the invention, a vector packaging plasmid containing a gene encoding the integrase-fusion protein, said gene being fused to the pol-polyprotein, is transfected into a lentiviral producer cell. Transcription and translation of the genes by the producer cell results in the production of a lentiviral vector particle, which contains the integrase-fusion protein fused to the pol-polyprotein. The integrase-fusion protein is then released from the pol-protein, inside the maturing vector particle.

In the method of the invention, foreign proteins are expressed from the vector packaging plasmid as in-frame fusions in the viral pol-gene. The desired protein is fused to the C-terminus of the viral integrase, which becomes released from the Pol polyprotein in the maturing virion. The integrase fusion protein may be cleaved from the precursor pol polyprotein by the viral protease.

Packaging of correctly sized integrase-fusion proteins into vector particles may occur without significant unspecific degradation. As integrase fusion proteins incorporate into new virions as parts of the large Gag-Pol-polyprotein, it may also be possible to package therapeutic proteins, such as cytotoxic proteins, into the virions. An additional advantage of this method is that virion-derived IN-fusion proteins are transported into the cells nucleus, as they form a part of the viral preintegration complex (PIC). However, if an extra protease cleavage site is introduced between the 3′ end of the integrase gene and the 5′ end of the foreign protein encoding gene, the packaged protein may be released into the target cell cytosol after vector uncoating. One important application of the method of the invention is that it enables the user to study the ability of different IN-fusion proteins to direct transgene integration in vitro and in vivo, in the context of a gene therapy grade third generation lentivirus vector. The present invention may also be used for transient delivery of foreign proteins into a variety of mammalian cells and to target IN-fusion proteins directly into a cell nucleus. This may be an important application for studying meganuclease-assisted homologous recombination in vivo, or the functionality of proapoptotic proteins.

IN-fusion protein baring LV vectors which carry the desired IN-fusion protein (instead of the viral integrase) were created. The proteins were packaged into LVX vectors and were composed of the wild-type human immunodeficiency virus type 1 (HIV-1) integrase. The HIV-1 integrase was fused from its C-terminus to the homing endonuclease I-Ppol, an N119A or an H78A mutated form of I-Ppol, the red fluorescent protein mCherry, or the proapoptotic cellular signaling protein p53. The integrase may also be fused to other mutated forms of I-Ppol, such as R61A. Further, IN may be fused to other homing endonuclease or meganuclease proteins, or to other proteins with therapeutic or cytotoxic properties.

I-Ppol has a natural target in the human genome and is able to recognize and cleave this well conserved sequence in the 28S rRNA gene. Constitutive expression of I-Ppol by human cells has been reported to lead to reduced cell survival or direct cell toxicity. The N119A mutation in I-Ppol has been reported to cause a dramatic reduction in the enzymes catalytic activity, but is not located in the areas which are important for DNA binding and specificity. The H78A mutation causes the activity of I-Ppol to decrease by about 50% compared to that of the wild type protein.

The following Example illustrates the invention.

Incorporation of IN-fusion Proteins into 3^{rd} Generation LV Vectors

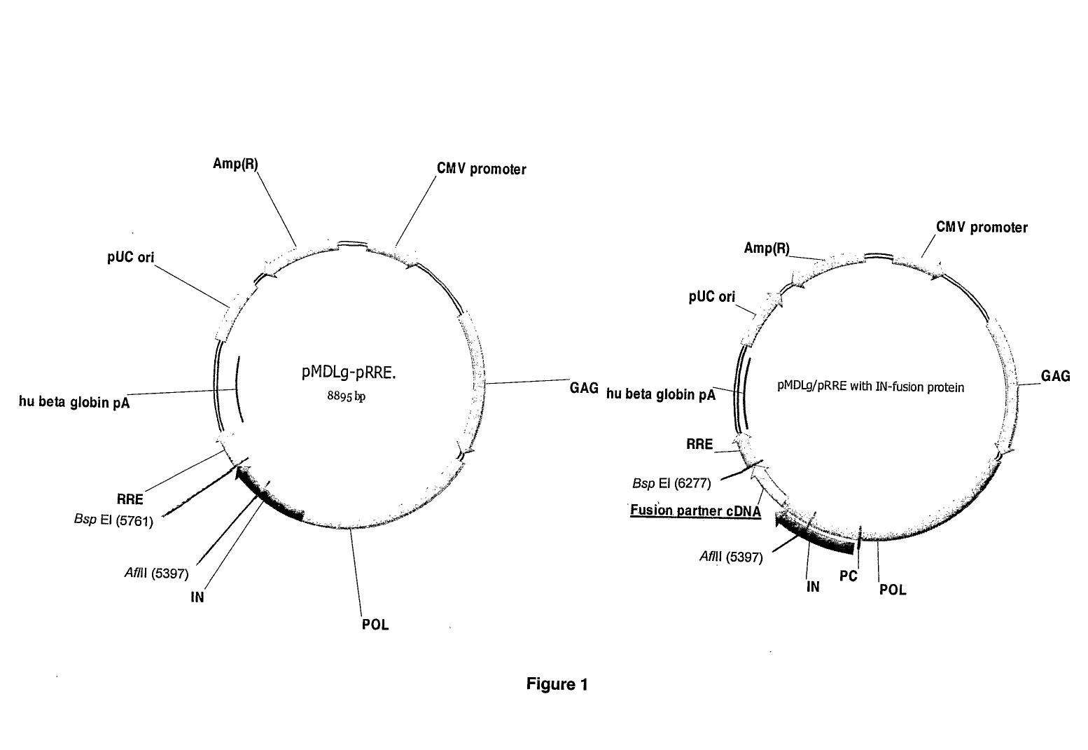

To evaluate whether functional IN-fusion proteins could be packaged into 3^{rd} generation lentiviral vectors without the need to first fuse the proteins into HIV-1 Vpr, versions of the HIV-1 vector packaging plasmid pMDLg/pRRE that contained an in-frame fusion of the different IN-fusion protein cDNAs in the pol-polyprotein gene, were constructed (FIG. 1). The natural protease cleavage site (PC) between the reverse transcriptase and integrase genes was preserved, as the N-terminal fragment of IN was not modified in the cloning strategy. Viruses were prepared by co-transfection of 4 plasmids (Follenzi & Naldini, 2002), one of which contained the different IN-fusion cDNAs in the pol-polyprotein gene, in place of the wild type IN (FIG. 1).

Also, LV (lentivirus) vectors containing a defective IN were prepared by using a packaging plasmid, wherein an inactivating D64V point mutation had been introduced in the IN coding region. These vectors were created to serve as a control when evaluating the enzymatic activity of the IN-fusion proteins. Correct packaging of IN-fusion proteins into vectors was verified by Western blotting. All fusion proteins were packaged as correct sized, and mostly undegraded, into the LV (lentivirus) vectors. Viruses were produced with good titers without modifications to the standard vector preparation protocol (Follenzi & Naldini, 2002).

In addition to vectors carrying a single type of IN molecule, vectors using the IN-fusion protein and either the defective or the wild type IN-containing packaging plasmids, were prepared. Mixed multimers of inactive (D64V mutated) IN mutants and the IN-fusion proteins, may be structurally more stable, and potentially more efficient in catalyzing transgene integration, than the IN-fusion proteins alone. The different IN-modified third generation LV vectors that were prepared are listed in Table 2.

### In Vitro Studies

To assess the possible cytotoxic effects of different IN-fusion protein containing vectors, human embryonic kidney 293 and HeLa cells were transduced, with a multiplicity of infection (M.O.I.) equal to 1, with the vectors LV-INIPpol, LV-INN119A, LV-GFP and LV-D64V. The morphology of the cells was followed for 2-5 days. Transfection of cells with MOI=1 resulted in GFP expression in approximately 55-95% of the HeLa cells at day 2 post infection. At day 2, the cells infected with LV-GFP, LV-D64V and LV-INN119A looked normal, but the cells infected with LV-INIPpol had already started to die. At day 3, most of the LV-INIPpol infected cells had detached from the culture plate and died, confirming the cytotoxic effects of an intranuclearly delivered active endonuclease I-Ppol. The cells remaining in the LV-INIPpol plates were GFP negative, and therefore not transduced with the vector.

The functionality of the LV vectors carrying a non-cytotoxic IN-fusion protein was assessed by their ability to catalyse transgene integration. The vector LV INmCherry was also characterised by the red fluorescence emitted by the IN-mCherry fusion protein. All the produced vectors containing a fusion protein IN were able to catalyse integration of the vector transgene in in vitro studies.

### DNA Constructs

The IN-fusion cDNAs were first cloned in pBluescript II. The IN cDNA was amplified by PCR using the primers 5′IN and 3′IN. The primer 3′IN was designed to delete the stop codon of IN and to introduce an in-frame XbaI site into the PCR fragment. Primers 5′Ppo and 3′Ppo were used to amplify I-Ppol. The primer 5′Ppo was designed to delete the start codon of the NLS sequence N-terminal to I-Ppol and to convert it into a SpeI site. Primer 3′Ppo was designed to contain a part of the I-Ppol cDNA ending in a stop codon, and a sequence of pMDLg/pRRE that ends in the BspEI site of the plasmid. Primers pRSET forward and 3′ mCherry BspEI were used to amplify mCherry from pRSET-B-mCherry, and they contained the same restriction endonuclease sites as the primers for I-Ppol. Primers p53(SpeI;NLS) forward and p53(NotI;Bspel,STOP) were used to amplify the cDNA of p53 from the plasmid p53pBacCaplRed (Hanna-Riikka Karkkainen). In addition to containing the same restriction sites as used with I-Ppol and mCherry before, the primer p53(SpeI;NLS) also introduced an NLS sequence into the p53 PCR product. Oligonucleotides used in PCR were purchased from Oligomer Oy, Helsinki and the sequences are shown in Table 1.

PCR fragments were purified by the Charge Switch® PCR Clean Up Kit (Invitrogen) and blunt end ligated/subcloned into the EcoRV site of pBluescript II to yield plasmids containing the fusion partners I-Ppol (pBS-IPpoI), mCherry (pBS-mCherry) and the HIV-1 integrase (pBS-IN). pBS-IPpol was digested with SpeI and the resulting fragment was gel extracted by PureLink Quick Gel Extraction Kit (Invitrogen). pBS-IN was linearised with XbaI and purified. The I-Ppol fragment was ligated into the XbaI site of pBS-IN to yield the plasmid pBS-INIPpol. The IN-mCherry fusion was created as described for I-Ppol. The purified p53 PCR fragment was digested with SpeI and NotI and ligated to pBS-IN digested with the same enzymes.

To construct the third generation lentivirus packaging plasmid pMDLg/pRRE with the IN-I-Ppol fusion gene, pBS-INIPpol was first digested with AfIII and BspEI, gel extracted and purified. pMDLg/pRRE was opened with AfIII and BspEI and the resulting plasmid fragment lacking most of the wt IN sequence was gel extracted and purified. The IN-I-Ppol gene fragment was ligated to pMDLg/pRRE cut with AfIII and BspEI to create the plasmid pMDLg/pRRE-Ppo (FIG. 1). The packaging plasmids containing the IN-mCherry and IN-p53 gene fusions were cloned from their parent pBluescript plasmids into pMDIg/pRRE as already described for IN-1-Ppol. The plasmid pMDLg/pRRE-N119A, containing an N119A mutation in the 1-Ppol gene, was created by site-directed mutagenesis using the QuikChangeR II XL Site-Directed Mutagenesis Kit (Stratagene La Jolla, Calif.) and the oligos N119A forward and N119A reverse. The plasmid pMDLg/pRRE-H78A, containing an H78A mutation in the I-Ppol gene, was created similarly using the primers H78A forward and reverse. An additional pMDLg/pRRE packaging plasmid was created where the IN gene was mutated to abolish its activity. This D64V mutation was done similarly by site-directed mutagenesis using the oligonucleotides D64V forward and D64V reverse. Clones were verified by sequencing.

### Preparation of IN-Modified HIV-1 Based Lentivirus Vectors

Vesicular stomatitis virus envelope (VSV-G) pseudotyped third generation HIV-1 based LV vectors were generated by the standard calcium phosphate mediated transfection of 293T cells (Follenzi & Naldini, 2002) with modifications in the usage of the plasmid pMDLg/pRRE. For every virus created, three of the four 3^{rd} generation LV packaging plasmids were always the same: pRSV-Rev, pMD2G and pLV-1, containing GFP cDNA as a transgene. The fourth 3^{rd} generation core packaging plasmids used were the modified IN-fusion containing pMDLg/pRRE plasmids, the wild type IN containing pMDLg/pRRE plasmid or the inactive IN harboring pMDLg/pRRE-D64V. Vectors were created using one of the three abovementioned modified pMDLg/pRRE plasmids at a time, or by mixing, in a 1:1 ratio, an IN-fusion containing pMDLg/pRRE plasmid with either wild type pMDLg/pRRE plasmid or pMDLg/pRRE-D64V. This packages both a fusion protein integrase and a wild type IN or an inactive integrase mutant into the same virus with the IN-fusion proteins (see table 5 for different LV vectors created). The virus titers were estimated by an enzyme-linked immunosorbent assay against the HIV-1 capsid (CA; p24) antigen and by determining the functional titer as GFP expression in HeLa cells.

### Western Blot

Correct incorporation of the different IN proteins into the LV vectors was verified by western blot. The IN proteins were detected using antisera to HIV-1 integrase, amino acids 23-34 (catalogue #757), obtained through NIH AIDS Research & Reference Reagent Program. The secondary antibody used was the Goat Anti-Rabbit IgG (H+L)-AP Conjugate (BIO-RAD).

### Cell Culture

Human embryonic kidney 293T cells (HEK 293T/17 ATCC® Number CRL-11268™) and HeLa cells (ATCC® Number: CCL-2™) were cultivated in DMEM supplemented with 10% Fetal Bovine Serum (FBS) at 37° C. in a 5% CO₂-containing humidified atmosphere.

In summary, the present invention enables the activity of IN-fusion proteins to be studied, specifically in the context of 3^{rd} generation LV vectors, which are devoid of unnecessary HIV-1 derived accessory proteins, such as Vif and Vpr. One advantage of the method is that equimolar amounts of IN-fusion proteins are produced and packaged into the vector particles along with other gag-pol gene products, whereas use of the Vpr-mediated trans-packaging strategy may lead to lower incorporation of heterologous proteins. Using this method, IN-fusion proteins are cleaved from the precursor Pol polyprotein at the natural RT-IN interphase by the viral protease, which may facilitate the correct release of the packaged proteins from the precursor protein.

Vpr-mediated trans-packaging may not be suitable for packaging toxic proteins into virions, whereas it has been shown herein that the expression of an active endonuclease protein, as a Pol fusion, is not detrimental to the vector producer cells, but efficiently kills vector transduced cells. The present invention may also be used to package foreign proteins, including cytotoxic or proapoptotic proteins, into HIV-1 vector particles. For example, the in vivo studying of meganuclease-assisted homologous recombination (HR) could be facilitated by the possibility to transduce cells with both the active meganuclease protein and the desired homology region containing linear transgene cassette. One advantage of studying meganuclease-assisted HR using the present invention is the nuclear localization of both the protein and the DNA substrate as parts of the viral PIC. The main features of the different IN-fusion protein incorporation strategies are summarized in Table 4.

Successful vector incorporation, using the method of the invention, may depend on the amino acid sequence of the foreign protein to be packaged. Using the method of the invention, large fusion proteins in the Pol-polyprotein are tolerated by the vector particle without reduction in particle formation or vector titer. Using the Vpr-linked approach, trans-packaging of up to 75 kDa-sized foreign proteins into PTNs was achieved (Link et al, 2006). This may reflect flexibility in the ability of lentivirus-derived particles to accept foreign proteins. Using the method of the invention, it is possible to incorporate 87 kDa-sized IN-P53 fusion proteins into LV vector particles.

The present invention shows the feasibility of packaging full-sized IN fusion proteins into third generation lentiviral vectors. The protein-packaging method of the invention enables the ability of IN-fusion proteins to target transgene integration into safe chromosomal sites to be studied, in the context of third generation lentivirus vectors.

Table 1 (below) shows DNA sequences of PCR primers used in construction of fusion proteins and in site-directed mutagenesis. In Table 1, a: restriction sites are underlined, with the identity of the restriction enzyme listed to the right; b: bold type letters denote the nucleotide substitutions introduced in the I-Ppol and IN cDNAs; and c: the letters in italics denote the nuclear localization signal added to the 5′ primers of I-Ppol and p53.

TABLE 1

| | | Restriction | Template; |
|---|---|---|---|
| Primer | Sequence^{a} | enzyme b | PCR product |
| 5′IN | CCTTAATTAAATGTTTTTAGATGGA | PacI | PLJS10, IN |
| | ATAGAT | | |
| | (SEQ ID No: 1) | | |
| 3′IN | GCTCTAGAATCCTCATCCTGTCTACT | XbaI | PLJS10, IN, cIN |
| | (SEQ ID No: 2) | | |
| 5'Ppo^{c} | ATTCACCACTAGTGCTCCAAAAAAA | SpeI, NLS | pCNPpo6: I-PpoI |
| | AAGCGC | | |
| | (SEQ ID No: 3) | | |
| 3′Ppo | GCTCCGGAATTCCATGTGTTATACCACAAA | BspEI | ″ |
| | GTGACTGCC | | |
| | (SEQ ID No: 4) | | |
| N119A | GGGAGTCACTAGACGACGCCAAAGGCAGA | | pMDLg/pRRE-Ppo; |
| forward^{b} | AACTGGTGCC | | pMDLg/pRRE- |
| | (SEQ ID No: 5) | | N119A |
| N119A | GGCACCAGTTTCTGCCTTTGGCGTCGTCTA | | pMDLg/pRRE-Ppo; |
| reverse^{b} | GTGACTCCC | | pMDLg/pRRE- |
| | (SEQ ID No: 6) | | N119A |
| H78A | CCACAGATGGGGATCCGCCACAGTCCCTT | | pMDLg/pRRE-Ppo; |
| forward^{b} | TTCTATTAGAACCGG | | pMDLg/pRRE-H78A |
| | (SEQ ID No: 7) | | |
| H78A | CCGGTTCTAATAGAAAAGGGACTGTGGCG | | pMDLg/pRRE-Ppo; |
| reverse^{b} | GATCCCCATCTGTGG | | pMDLg/pRRE-H78A |
| | (SEQ ID No: 8) | | |
| D64V | GCCCAGGAATATGGCAGCTAGTGTGTACA | | pMDLg/pRRE; |
| forward^{b} | CATTTAGAAGG | | pMDLg/pRRE-D64V |
| | (SEQ ID No: 9) | | |
| D64V | CCTTCTAAATGTGTACACACTAGCTGCCAT | | pMDLg/pRRE; |
| reverse^{b} | ATTCCTGGGC | | pMDLg/pRRE-D64V |
| | (SEQ ID No: 10) | | |
| p53 | TGCAGGCGAAACTAGTGCTCCAAAAAAAAA | SpeI, NLS | p53pBacCapIRed; |
| forward^{c} | GCGCAAAGTGGAGGAGCCGCAGTCAG | | p53 |
| | (SEQ ID No: 11) | | |
| p53 | GATGATGGTGGTGTGCGGCCGCTCCGGAT | NotI, BspEI | p53pBacCapIRed; |
| reverse | TAGTCTGAGTCAGGCCC | | p53 |
| | (SEQ ID No: 12) | | |
| pRSET | AAGGAACTAGTGTGAGCAAGGGCGAGGAG | SpeI | pRSET-B-mCherry; |
| forward | (SEQ ID No: 13) | | mCherry |
| 3′mCherry | TCCGGATTACTTGTACAGCTCGTCCAT | BspEI | pRSET-B-mCherry; |
| BspEI | (SEQ ID No: 14) | | mCherry |

Table 2 (below) lists vectors containing different IN modifications. The vectors can be used individually or mixed in any combination.

TABLE 2

| Vector | IN-modification | Vector Characteristics |
|---|---|---|
| LV-GFP | None | Wild type vector |
| LV-D64V | D64V mutation | Integration deficient LV |
| | | vector |
| LV-INIPpol | C-terminally fused to | Carries an active I-Ppol |
| | I-Ppol | endonuclease fused to IN |
| LV-INN119A | C-terminally fused to | Carries an inactive I-Ppol |
| | N119A mutated I-Ppol | endonuclease fused to IN |
| LV-INH78A | C-terminally fused to | Carries an 50% active |
| | H78A mutated I-Ppol | I-Ppol endonuclease fused |
| | | to IN |
| LV-INp53 | C-terminally fused to | Carries an IN-p53 fusion |
| | p53 | protein |
| LV- | C-terminally fused to | Carries a red fluorescent |
| INmCherry | mCherry | IN-mCherry fusion protein |
| LV-D64VPpo | D64V mutation + fused to | Carries an inactive IN fused |
| | I-Ppol | to active I-Ppol |
| | | endonuclease |
| LV- | D64V mutation + fused to | Carries an inactive IN fused |
| D64VN119A | N119A mutated I-Ppol | to inactive I-Ppol |
| | | endonuclease |
| LV-D64Vp53 | D64V mutation + fused to | Carries an inactive IN fused |
| | p53 | to p53 |
| LV- | D64V mutation + fused to | Carries an inactive IN fused |
| D64VmCherry | mCherry | to red fluorescent mCherry |
| | | protein |

Table 3 is a comparison of the different approaches to incorporate IN-fusion proteins into HIV-1 derived vectors or viruses. Env, virus envelope protein; VSV-G, Vesicular stomatitis virus G protein; PR, Lentivirus protease; PC, protease cleavage site; LVV, lentivirus vector.

TABLE 3

| | | | 3^{rd} Generation LV with |
|---|---|---|---|
| | Trans-packaging | Genomic fusion | IN-fusion protein |
| Production method of | Cotransfection of three | Transfection of | Cotransfection of four |
| IN-fusion protein | constructs into | the HIV-1 | constructs into |
| containing virus/vector | producer cells | genomic clone | producer cells |
| | | containing the IN- | |
| | | fusion gene | |
| Packaging construct | HIV-1 genomic clone | HIV-1 genomic | Conditional packaging |
| (source of structural | with Env (and Vpr) | clone | plasmid pMDLg/pRRE |
| genes) | deletion/substitution | | expressing only Env |
| | | | and Pol |
| Source of IN-fusion | A separate Vpr-PC-IN | HIV-1 genomic | Modified conditional |
| protein | expression plasmid | clone | packaging plasmid |
| | | | pMDLg/pRRE |
| Ease of IN-fusion | Good; separate plasmid | Poor; overlapping | Good; no overlapping |
| protein sequence | encoding for Vpr-IN- | regulatory and | regulatory and coding |
| addition into packaging | fusion proteins | coding regions in | regions in the |
| construct | | the genomic | packaging plasmid |
| | | clone | |
| Source of viral envelope | Separate MLV Env/ | HIV-1 genomic | Separate VSV-G |
| (pseudotyping) | VSV-G expression | clone | expression plasmid |
| | plasmid | | (pMD2G) |
| PR mediated release of | Partial/imperfect | | Effective release from |
| IN-fusion proteins from | cleavage from Vpr-PC- | | Pol-polyprotein |
| packaging construct | construct | | |
| The form of IN (-fusion) | Partly in an inactive | | Full-sized IN-fusion |
| proteins inside the virion | form if remains fused to | | protein |
| | Vpr, occurrence of | | |
| | unspecific IN-protein | | |
| | degradation | | |
| Presence of wt or | Yes | No | No/Yes if desired |
| mutated IN protein in the | | | |
| virion | | | |
| Source of viral RNA | HIV-1 genomic clone | HIV-1 genomic | pLV-GFP-WPRE-SIN |
| genome/vector RNA | with Env (and Vpr) | clone | containing self- |
| (=transfer construct) | deletion/substitution | | inactivating (SIN) |
| | | | LTR's and no viral |
| | | | genes |
| Ease of changing the | Poor | Poor | Good |
| transgene in the transfer | | | |
| construct | | | |
| Additional safety | Env (and Vpr) deleted | None | Non-overlapping |
| features | provirus construct | | production constructs, |
| | | | no viral gene products |
| | | | in transfer construct, • |
| | | | SIN LTR's |
| Vector/virus generation | Closest to first | Almost wt, poor | Third generation, |
| and safety | generation, poor safety | safety | safest LVV form to |
| | | | date |

### REFERENCES

Bushman and Miller. 1997. J. Virol. 71:458-464.
Bushman (1994). Proc. Natl. Acad. Sci. USA 91: 9233-9237.
Bushman (1995). Science: 267: 1443-1444.
Bushman (2003). Cell 115:135-138.
Fletcher et al, EMBO J. 16:5123-5138.
Follenzi and Naldini (2002). Methods Enzymol. 346:454-65.
Goulaouic and Chow (1996). J. Virol. 70: 37-46
Holmes-Son and Chow. 2002. Mol. Ther. 5:360-370.
Katz et al, (1996). Virology. 217(1):178-90.
Kondo et al, 1995. J. Virol. 69:2759-2764.
Link et al, (2006) Nucleic Acids Res. 34(2):e16.
Tan et al, 2006. J. Virol. 80:1939-48.
Wu et al, 1995. J. Virol. 69:3389-3398.

1. A method for incorporating an integrase-fusion protein into a third-generation lentivirus vector, comprising:
(i) transfecting a vector packaging plasmid into a lentivirus producer cell, wherein the vector packaging plasmid contains a gene encoding the integrase-fusion protein, said gene being fused to the pol-polyprotein gene;
(ii) transcription and translation of the genes by the lentivirus producer cell to produce a lentiviral vector particle; and
(iii) inside the vector particle, release of the integrase-fusion protein from the pol-polyprotein.

2. The method according to claim 1, wherein the integrase fusion protein comprises a meganuclease protein or homing endonuclease protein.

3. The method according to claim 1, wherein the integrase fusion protein comprises a marker protein.

4. The method according to claim 3, wherein the marker protein comprises mCherry, I-Ppol, N119A, R61A or H78A.

5. The method according to any preceding claim 1, wherein the integrase fusion protein comprises a therapeutic or proapoptotic protein.

6. The method according to claim 5, wherein the therapeutic or proapoptotic protein is p53.

7. The method according to claim 5, wherein the therapeutic or proapoptotic protein is a cytotoxic protein.

8. The method according to claim 1, wherein the integrase-fusion protein comprises D64V.
